(19) United States
(12) Patent Application Publication
(10) Pub. No.: US 2018/0056019 A1
(43) Pub. Date: Mar. 1, 2018

(54) NASOGASTRIC TUBE

(71) Applicant: Jang-Jier TSENG, Taipei (TW)

(72) Inventor: Jang-Jier TSENG, Taipei (TW)

(21) Appl. No.: 15/680,563

(22) Filed: Aug. 18, 2017

(30) Foreign Application Priority Data
Aug. 23, 2016 (TW) 105126973

(51) Int. Cl. A61M 16/04; A61J 15/00

(52) U.S. Cl. CPC A61M 16/0461; A61J 15/0023; A61M 25/0662; A61J 15/0026; A61J 15/0003

(57) ABSTRACT

A nasogastric tube includes an outer tube and an inner tube. The outer tube has a receiving space and at least one opening, the receiving space is formed within the outer tube, and the opening is connected to the receiving space and formed at a sidewall of an end of the outer tube. The inner tube is longitudinally mounted in the receiving space to pass through the end of the outer tube and has an accommodating space and at least one through hole. The accommodating space is formed within the inner tube, and the through hole is connected to the accommodating space and formed at a sidewall of the inner tube passing through the end of the outer tube.

### CROSS REFERENCE

This non-provisional application claims priority from Taiwan Patent Application No. 105126973, filed on Aug. 23, 2016, the content thereof is incorporated by reference herein.

### FIELD OF THE INVENTION

The present invention is directed to a feeding device, and more particularly to a nasogastric tube.

### BACKGROUND OF THE INVENTION

Enteral nutrition support is a medical method wherein a man-made conduit, e.g. a nasogastric tube, an orogastric tube, or a gastrostomy tube, is placed in a subject, and then food is supplied to the stomach via this conduit. In such a manner, the food does not contact the mouth, the pharynx, the larynx, or the esophagus to avoid mucous irritation, respiratory tract infection, choke, or vomiting reflex. In clinic, enteral nutrition support provides supportive treatment for patients losing normal eating, having insufficient feeding, suffered from a disease related to mouth, pharynx, larynx, esophagus, or a stroke, or in a coma. Therefore, various nutrition ingredients can be delivered to patients periodically with a predetermined amount to satisfy their physiological requirements.

The currently used nasogastric tube is placed in a subject for a period of no more than one month. However, most patients must be received enteral nutrition support for several months, or even for several years. Therefore, nasogastric tube substitution is required for patients every month. During the non-substitution period of each month, there exist some scenarios leading to nasogastric tube displacement, such as sneezing, coughing, involuntary choking with something, or intentional extubation. In case of nasogastric tube displacement, nasogastric tube replacement is required. During nasogastric tube placement or replacement, nasogastric tube insertion into the bronchus must be avoided, and nasogastric tube entrance into the stomach is ensured. Accordingly, nasogastric tube placement and replacement must be executed by a health care professional. That is, when nasogastric tube displacement occurs, the patient must be taken to a hospital or a clinic for nasogastric tube replacement. It is a fact that nasogastric tube displacement may take place for several times within the non-substitution period of each month. In the case, enteral nutrition support is a burden for a patient, the patient's family, and a health care professional.

The currently-employed process for nasogastric tube placement is uncomfortable for patients with clear consciousness, which contributes to that the nasogastric tube can trigger coughing or choking, and such reactions can further lead to nasal mucus secretion from a nasal cavity or saliva secretion. A health care professional experienced with nasogastric tube placement still has to ask a patient to swallow, and insert the nasogastric tube through the epiglottis into the oesophagus at the instant of swallowing. After placement, the nasogastric tube may hurt mucous membranes or elicit feeling of foreign substances, ulcers, or infection. Furthermore, the currently-used nasogastric tube is not suitable for patients losing swallow reflex or cough reflex, and patients with craniofacial fractures.

The currently-used nasogastric tube, because of its size and material, has poor flexibility. When the nasogastric tube is placed in a subject for a long period, it can result in lower esophageal sphincter dysfunction. It is reported that patients without cough reflex or with facial paralysis have the higher probability of suffering from gastroesophageal reflux, substance reflux from the stomach to the pharynx through the lower esophageal sphincter and the space between the nasogastric tube and the mucous membranes. In addition, the higher probability of suffering from gastroesophageal reflux means higher probability of suffering from aspiration pneumonia, which has a mortality rate of 40%.

As above, there is a need to develop a simple nasogastric tube which can provide painless nasogastric tube placement and easy substitution for nasogastric tubes, and avoid aspiration pneumonia.

### SUMMARY OF THE INVENTION

An objective of the present invention is to provide a nasogastric tube, which includes an outer tube and an inner tube. The outer tube has a receiving space and at least one opening, the receiving space is formed within the outer tube, and the opening is connected to the receiving space and formed at a sidewall of an end of the outer tube. The inner tube is longitudinally mounted in the receiving space to pass through the end of the outer tube and has an accommodating space and at least one through hole. The accommodating space is formed within the inner tube, and the through hole is connected to the accommodating space and formed at a sidewall of the inner tube passing through the end of the outer tube.

Another objective of the present invention is to provide a method for feeding a subject, and the method includes the steps of: (a) providing a nasogastric tube comprising an outer tube and an inner tube, the outer tube having a receiving space and at least one opening, the receiving space formed within the outer tube, the opening connected to the receiving space and formed at a sidewall of an end of the outer tube, the inner tube longitudinally mounted in the receiving space to pass through the end of the outer tube and having an accommodating space and at least one through hole, the accommodating space formed within the inner tube, and the through hole connected to the accommodating space and formed at a sidewall of the inner tube passing through the end of the outer tube; (b) inserting the nasogastric tube into a subject; (c) introducing gas into the accommodating space of the inner tube; (d) determining whether the gas is positioned in a stomach of the subject through the through hole, if no, and then withdrawing the nasogastric tube from the subject and performing step (b); if yes, and then moving the nasogastric tube to allow the opening to get into the stomach; and (e) supplying a fluidic substance to the stomach through the receiving space and the opening in sequence.

Another yet objective of the present invention is to provide a method for nasogastric tube substitution, and the method includes the steps of: (a) providing a first nasogastric tube and a second nasogastric tube, each nasogastric tube comprising an outer tube and an inner tube, the outer tube having a receiving space and at least one opening, the receiving space formed within the outer tube, the opening connected to the receiving space and formed at a sidewall of an end of the outer tube, the inner tube longitudinally mounted in the receiving space to pass through the end of the outer tube and having an accommodating space and at least one through hole, the accommodating space formed within the inner tube, and the through hole connected to the accommodating space and formed at a sidewall of the inner tube passing through the end of the outer tube; (b) inserting the first nasogastric tube into a subject to allow the opening of the first nasogastric tube to get into a stomach of the subject; (c) outwardly moving the first nasogastric tube at a predetermined distance relative to the subject; (d) inserting the second nasogastric tube into the receiving space of the first nasogastric tube to allow the through hole of the second nasogastric tube through the opening of the first nasogastric tube in the stomach; and (e) withdrawing the first nasogastric tube from the subject.

### BRIEF DESCRIPTION OF THE DRAWINGS

FIG. 1 is a longitudinal cross-section view of a nasogastric tube according to an embodiment;

FIG. 2 is a top schematic view of the nasogastric tube;

FIG. 3 is a bottom schematic view of the nasogastric tube;

FIG. 4A is a schematic view showing the use of a nasogastric tube according to an embodiment; and

FIG. 4B is a schematic view showing the substitution of nasogastric tubes according to an embodiment.

### DETAILED DESCRIPTION OF THE INVENTION

The detailed description and preferred embodiments of the invention will be set forth in the following content, and provided for people skilled in the art so as to understand the characteristics of the invention.

### Embodiment 1

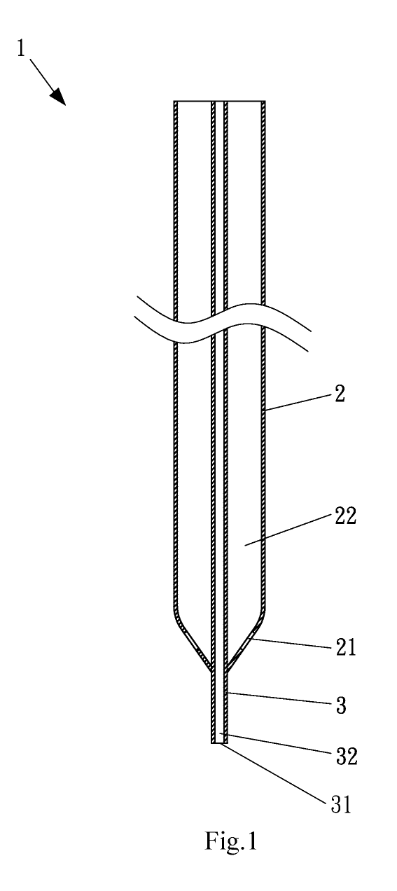

A nasogastric tube (1) of an embodiment is depicted in FIG. 1, and includes an outer tube (2) and an inner tube (3).

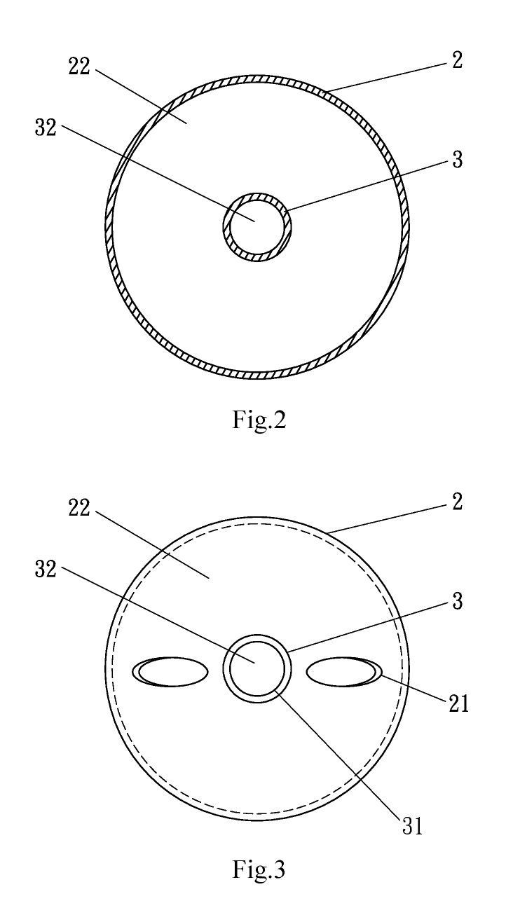

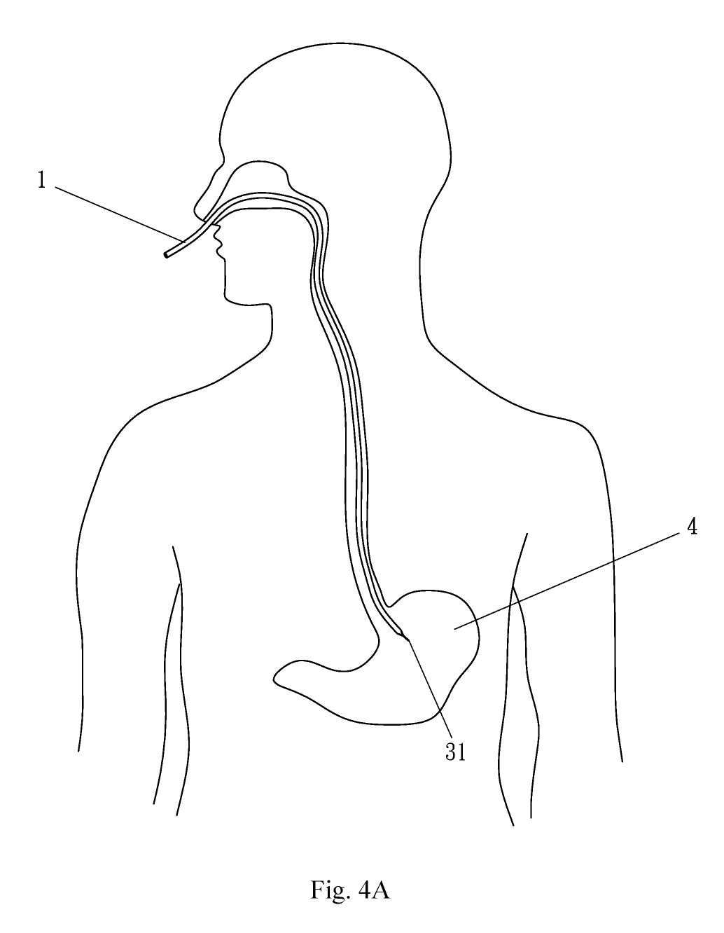

As shown in FIGS. 2-3, the outer tube (2) has a receiving space (22) and at least one opening (21). Preferably, the outer tube (2) is made of silicone, polyurethane, polyethylene, polyvinyl chloride, high density polyethylene, or polyolefin thermoplastic elastomer. Also preferably, the outer tube (2) has a wall thickness of 0.001 mm to 0.3 mm. The receiving space (22) is formed within the outer tube (2). The opening (21) is connected to the receiving space (22) and formed at a sidewall of an end of the outer tube (2).

The inner tube (3) is longitudinally mounted in the receiving space (22) to pass through the end of the outer tube (2) and has an accommodating space (32) and at least one through hole (31). Preferably, the inner tube (3) is made of silicone, polyurethane, or polyvinyl chloride. Also preferably, the inner tube (3) is coaxially arranged with the outer tube (2). Further preferably, the inner tube (3) is integrally connected to the outer tube (2). The accommodating space (32) is formed within the inner tube (3). The through hole (31) is connected to the accommodating space (32) and formed at a sidewall of the inner tube (3) passing through the end of the outer tube (2).

As above, the nasogastric tube (1) can be used for feeding a subject with a fluidic substance, such as a fluidic food or a fluidic nutriment. During feeding, the nasogastric tube (1) is firstly inserted into the subject to allow the through hole (31) in the stomach. Since the inner tube (3) is substantially rigid, it is easy to insert the nasogastric tube (1) into the subject and direct the through hole (31) into the stomach. Additionally, gas can be introduced into the accommodating space (32). When the gas enters the stomach, it is ascertainable that the through hole (31) is in the stomach. On the other hand, when the through hole (31) is in the stomach, the gas in the stomach can be withdrawn through the accommodating space (32). Furthermore, the integral connection of the inner tube (3) to the outer tube (2) can make them simultaneously be placed at the same position of the subject with the nasogastric tube (1) inserted into the subject.

Next, the fluidic substance is supplied to the receiving space (22) to enter the stomach through the opening (21). Since the outer tube (2) is substantially yielding, the outer tube (2) can fit the shapes of nasal cavity, the pharynx, the larynx, and deform with the peristalsis of oesophagus. In addition, the outer tube (2) can adhere to the mucous membrane via the mucosal secretion. Thus, the nasogastric tube (1) not only can provide low discomfort, but also can avoid hurting the mucous membrane when it passes through the nasal cavity, the mouth, the pharynx, the larynx, or the oesophagus, or when it is placed in the subject for a long period. Also, another nasogastric tube (1) can be easily inserted through the one placed in the subject, and thus the probability of inserting each nasogastric tube into the trachea or the lung can be lowered. Furthermore, the nasogastric tube (1) can reduce the incidence of aspiration pneumonia resulted from any traditional nasogastric tube.

### Embodiment 2

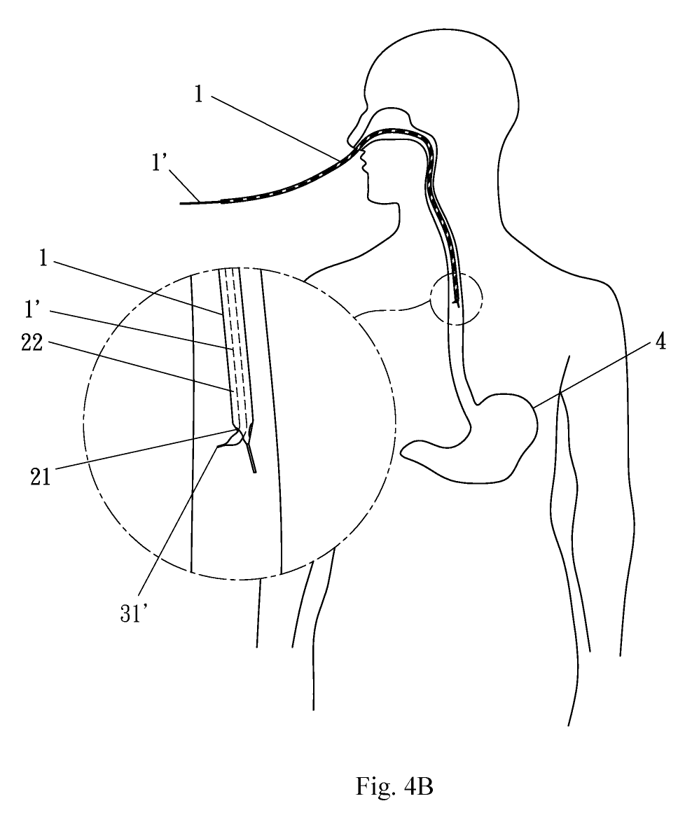

A method for feeding a subject of an embodiment is described herein. Firstly, a nasogastric tube having a structure as described above is provided. After which, the nasogastric tube is inserted into a subject. At this moment, it is doubtable whether the nasogastric tube is placed in the esophagus. For this reason, gas is introduced into the accommodating space of the inner tube, and then it is determined whether the gas is positioned in the stomach through the through hole. If the gas is not positioned in the stomach through the through hole, it is indicated that the nasogastric tube is not in the esophagus. Therefore, the nasogastric tube is withdrawn from the subject and then the nasogastric tube insertion into the subject is performed again. If the gas is positioned in the stomach through the through hole, it is indicated that the nasogastric tube is in the esophagus. Therefore, the nasogastric tube (1) is moved to allow the opening (21) to get into the stomach (4) (FIG. 4A). Afterward, a fluidic substance, e.g. a fluidic food or a fluidic nutriment, is supplied to the stomach through the receiving space and the opening in sequence.

Preferably, after the nasogastric tube moving and before the fluidic substance supplying, a gastric residual of the subject is acquired through the through hole and the accommodating space in sequence, and then a pH value thereof is measured. In this way, it can be rechecked whether the through hole is in the stomach.

Preferably, after the instant fluidic substance supplying and before the next fluidic substance supplying, another gastric residual of the subject is acquired through the through hole and the accommodating space in sequence, and then total volume thereof is measured to determine whether the instant fluidic substance is digested well already. If total volume of the gastric residual is low, it is revealed that the instant fluidic substance is digested well already. Therefore, the next fluidic substance supplying is optionally performed. If total volume of the gastric residual is high, it is revealed that the fluidic substance is not digested well already. Therefore, the next fluidic substance supplying is neglected.

### Embodiment 3

A method for nasogastric tube substitution of an embodiment is described herein. Firstly, a first nasogastric tube and a second nasogastric tube are provided and each has a structure as described above. Subsequently, the first nasogastric tube is inserted into a subject to allow the opening thereof in the stomach. Preferably, the first nasogastric tube insertion into the subject is performed with reference to the foregoing method. Next, the first nasogastric tube is outwardly moved at a predetermined distance relative to the subject. Preferably, the predetermined distance is of 20 cm to 40 cm. After, the second nasogastric tube (1′) is inserted into the receiving space (22) of the first nasogastric tube (1) to allow the through hole (31′) of the second nasogastric tube (1′) through the opening (21) of the first nasogastric tube (1) in the stomach (4) (FIG. 4B). Finally, the first nasogastric tube is withdrawn from the subject.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A nasogastric tube, comprising:
an outer tube having a receiving space and at least one opening, the receiving space formed within the outer tube, and the opening connected to the receiving space and formed at a sidewall of an end of the outer tube; and
an inner tube longitudinally mounted in the receiving space to pass through the end of the outer tube and having an accommodating space and at least one through hole, the accommodating space formed within the inner tube, and the through hole connected to the accommodating space and formed at a sidewall of the inner tube passing through the end of the outer tube.

2. The nasogastric tube as claimed in claim 1, wherein the inner tube is coaxially arranged with the outer tube.

3. The nasogastric tube as claimed in claim 1, wherein the inner tube is integrally connected to the outer tube.

4. The nasogastric tube as claimed in claim 1, wherein the outer tube is made of silicone, polyurethane, polyethylene, polyvinyl chloride, high density polyethylene, or polyolefin thermoplastic elastomer, and the inner tube is made of silicone, polyurethane, or polyvinyl chloride.

5. The nasogastric tube as claimed in claim 1, wherein the outer tube has a wall thickness of 0.001 mm to 0.3 mm.

6. A method for feeding a subject, comprising:
(a) providing a nasogastric tube comprising an outer tube and an inner tube, the outer tube having a receiving space and at least one opening, the receiving space formed within the outer tube, the opening connected to the receiving space and formed at a sidewall of an end of the outer tube, the inner tube longitudinally mounted in the receiving space to pass through the end of the outer tube and having an accommodating space and at least one through hole, the accommodating space formed within the inner tube, and the through hole connected to the accommodating space and formed at a sidewall of the inner tube passing through the end of the outer tube;
(b) inserting the nasogastric tube into a subject;
(c) introducing gas into the accommodating space of the inner tube;
(d) determining whether the gas is positioned in a stomach of the subject through the through hole, if no, and then withdrawing the nasogastric tube from the subject and performing step (b); if yes, and then moving the nasogastric tube to allow the opening to get into the stomach; and
(e) supplying a fluidic substance to the stomach through the receiving space and the opening in sequence.

7. The feeding method as claimed in claim 6, between steps (d) and (e) further comprising:
(d1) acquiring a gastric residual of the subject through the through hole and the accommodating space in sequence; and
(d2) measuring a pH value of the gastric residual.

8. The feeding method as claimed in claim 6, after step (e) further comprising:
(e1) acquiring a gastric residual of the subject through the through hole and the accommodating space in sequence; and
(e2) measuring total volume of the gastric residual to determine whether the fluidic substance is digested well already, if yes, and then optionally supplying another fluidic substance to the stomach through the receiving space and the opening in sequence; if no, and then neglecting the next fluidic substance supplying step.

9. The feeding method as claimed in claim 6, wherein the fluidic substance is a fluidic food or a fluidic nutriment.

10. The feeding method as claimed in claim 6, wherein the inner tube is coaxially arranged with the outer tube.

11. The feeding method as claimed in claim 6, wherein the inner tube is integrally connected to the outer tube.

12. The feeding method as claimed in claim 6, wherein the outer tube is made of silicone, polyurethane, polyethylene, polyvinyl chloride, high density polyethylene, or polyolefin thermoplastic elastomer, and the inner tube is made of silicone, polyurethane, or polyvinyl chloride.

13. The feeding method as claimed in claim 6, wherein the outer tube has a wall thickness of 0.001 mm to 0.3 mm.

14. A method for nasogastric tube substitution, comprising:
(a) providing a first nasogastric tube and a second nasogastric tube, each nasogastric tube comprising an outer tube and an inner tube, the outer tube having a receiving space and at least one opening, the receiving space formed within the outer tube, the opening connected to the receiving space and formed at a sidewall of an end of the outer tube, the inner tube longitudinally mounted in the receiving space to pass through the end of the outer tube and having an accommodating space and at least one through hole, the accommodating space formed within the inner tube, and the through hole connected to the accommodating space and formed at a sidewall of the inner tube passing through the end of the outer tube;
(b) inserting the first nasogastric tube into a subject to allow the opening of the first nasogastric tube in a stomach of the subject;
(c) outwardly moving the first nasogastric tube at a predetermined distance relative to the subject;
(d) inserting the second nasogastric tube into the receiving space of the first nasogastric tube to allow the through hole of the second nasogastric tube through the opening of the first nasogastric tube in the stomach; and
(e) withdrawing the first nasogastric tube from the subject.

15. The substitution method as claimed in claim 14, wherein the predetermined distance is of 20 cm to 40 cm.

16. The substitution method as claimed in claim 14, wherein the inner tube is coaxially arranged with the outer tube.

17. The substitution method as claimed in claim 14, wherein the inner tube is integrally connected to the outer tube.

18. The substitution method as claimed in claim 14, wherein the outer tube is made of silicone, polyurethane, polyethylene, polyvinyl chloride, high density polyethylene, or polyolefin thermoplastic elastomer, and the inner tube is made of silicone, polyurethane, or polyvinyl chloride.

19. The substitution method as claimed in claim 14, wherein the outer tube has a wall thickness of 0.001 mm to 0.3 mm.
